(19) United States
(12) Patent Application Publication
(10) Pub. No.: US 2024/0423693 A1
(43) Pub. Date: Dec. 26, 2024

(54) SYSTEMS, DEVICES AND METHODS RELATING TO CONTROLLING HEATING IN A RADIOFREQUENCY TREATMENT PROCEDURE

(71) Applicant: AVENT, INC., Alpharetta, GA (US)

(72) Inventors: Lisa M. MCGREGOR, Alpharetta, GA (US); Michael Douglas BROWN, Alpharetta, GA (US); Jennifer J. BARRETT, Alpharetta, GA (US)

(21) Appl. No.: 18/711,058

(22) PCT Filed: Nov. 18, 2022
(86) PCT No.: PCT/US2022/050391

§ 371 Date: May 16, 2024

Related U.S. Application Data
(60) Provisional application No. 63/281,249, filed on Nov. 19, 2021.

(51) Int. Cl. A61B 18/12; A61B 18/00; A61B 18/16

(52) U.S. Cl. CPC A61B 18/1206; A61B 18/16; A61B 2018/00577; A61B 2018/00648; A61B 2018/00702; A61B 2018/0072; A61B 2018/00791; A61B 2018/00875; A61B 2018/167

(57) ABSTRACT

Systems and methods for controlling a radiofrequency (RF) treatment are provided. In some embodiments, the method can include: performing, using a programmable controller, at least the following functions of: measuring an electrical energy flow through a grounding pad that is in contact with at least a portion of skin of a subject's skin; determining, based on the measured electrical energy flow, a heating factor that represents a measure of heat absorbed by the subject over a period of time in response to treatment of the subject with an electrical radiofrequency probe; and comparing the heating factor to a predetermined threshold. In response to determining from the comparison that the heating factor is approaching or greater than the predetermined threshold value, the method can include disabling the radiofrequency generator or changing the electrical energy flow such that the controller regulates the electrical energy flow using a closed loop feedback system.

### CROSS-REFERENCE TO RELATED APPLICATIONS

This application claims the benefit of and priority to U.S. Provisional Patent App. No. 63/281,249, filed Nov. 19, 2021, which is incorporated herein by reference in its entirety.

### BACKGROUND

Lower back injuries and chronic joint pain are major health problems resulting not only in debilitating conditions for the patient, but also in the consumption of a large proportion of funds allocated for health care, social assistance and disability programs. In the lower back, disc abnormalities and pain may result from trauma, repetitive use in the workplace, metabolic disorders, inherited proclivity, and/or aging. The existence of adjacent nerve structures and innervation of the disc are very important issues in respect to patient treatment for back pain. In joints, osteoarthritis is the most common form of arthritis pain and occurs when the protective cartilage on the ends of bones wears down over time.

A minimally invasive technique of delivering high-frequency electrical current has been shown to relieve localized pain in many patients. Generally, the high-frequency current used for such procedures is in the radiofrequency (RF) range, i.e., between 100 kHz and 1 GHz and more specifically between 300-600 kHz. The treatment of pain using high-frequency electrical current has been applied successfully to various regions of patients' bodies suspected of contributing to chronic pain sensations. In addition to creating lesions in neural structures, application of radiofrequency energy has also been used to treat tumors throughout the body.

The RF electrical current is typically delivered from a generator via connected electrodes that are placed in a patient's body, in a region of tissue that contains a neural structure suspected of transmitting pain signals to the brain. The electrodes generally include one of more probes defining an insulated shaft with an exposed conductive active electrode tip to deliver the radiofrequency electrical current. Tissue resistance to the current causes heating of tissue adjacent resulting in the coagulation of cells (at a temperature of approximately 45° C. for small unmyelinated nerve structures) and the formation of a lesion that effectively denervates the neural structure in question. Denervation refers to affecting a neural structure's ability to transmit signals and usually results in the complete inability of a neural structure to transmit signals, thus removing the pain sensations.

To extend the size of a lesion, radiofrequency treatment may be applied in conjunction with a cooling mechanism, whereby a cooling means is used to reduce the temperature of the tissue near an energy delivery device, allowing a higher voltage to be applied without causing an unwanted increase in local tissue temperature. The application of a higher voltage allows regions of tissue further away from the energy delivery device to reach a temperature at which a lesion can form, thus increasing the size/volume of the lesion compared to conventional (non-cooling) radiofrequency treatments, where the larger size/volume of the lesion can increase the probability of success of ablating a target nerve. Cooled radiofrequency ablation is achieved by delivering, in a closed-loop circulation, cooling fluid (e.g., sterile water) via a peristaltic pump through the probe/active electrode. The cooling fluid continuously transfers heat away from the active electrode, allowing the electrode-tissue interface temperature to be maintained at a level that does not char or significantly desiccate the surrounding tissue, which is the primary limitation of conventional radiofrequency ablation. As a result, more radiofrequency energy can be delivered to the tissue, creating a lesion having a larger volume/size compared to a lesion created by conventional radiofrequency ablation.

However, if excessive electrical energy is delivered in a time period, it can cause tissue heating that can result in the aforementioned unwanted charring or desiccation of tissue.

It is with respect to these and other considerations that the various embodiments described below are presented.

### SUMMARY

In some aspects, the present disclosure relates to systems, devices and methods relating to controlling heating in an RF treatment procedure.

In one aspect, the present disclosure relates to a method for radiofrequency (RF) treatment, which, in one embodiment, includes performing, using a programmable controller, at least the following functions: measuring an electrical energy flow through a grounding pad, where the grounding pad is in contact with at least a portion of skin of a subject's skin, and where the electrical energy is delivered to a treatment site of the subject, from a radiofrequency (RF) generator by at least one radiofrequency probe; determining, based on the measured electrical energy through the grounding pad, a heating factor, where the heating factor represents a measure of heat absorbed by the subject over a predetermined period of time in response to the delivery of the electrical energy to the subject by the at least one radiofrequency probe; comparing the heating factor to a predetermined threshold value associated with damage to the subject outside of the treatment site, and in response to determining, from the comparison, that the heating factor is approaching or greater than the predetermined threshold value, performing at least one of: disabling the radiofrequency generator for at least a predetermined period of time; or increasing or decreasing the electrical energy flow, such that the controller regulates the electrical energy flow in accordance with a closed loop feedback system.

In one embodiment the heating factor is a total heating factor that represents a running average of a plurality of incremental heating factors.

In one embodiment, the incremental heating factors are recorded at regular intervals.

In one embodiment, the method includes measuring the electrical energy flow through the at least one radiofrequency probe, where the at least one radiofrequency probe is in contact with at least a first portion of the skin of the subject at the treatment site; and disabling the at least one radiofrequency ablation probe when the measured electrical energy flow from the at least one radiofrequency ablation probe is different from the electrical energy flow through the grounding pad.

In one embodiment, the at least one radiofrequency probe is a plurality of radiofrequency probes that are in contact with a plurality of locations at the treatment site, and where a sum of the electrical energy flow through each of the radiofrequency probes is compared to the electrical energy flow through the grounding pad; and where the method includes: disabling all of the radiofrequency probes in response to determining that the sum of the electrical energy flow through the radiofrequency probes is different from the electrical energy flow through the grounding pad.

In one embodiment, the method includes calculating an expected heating factor that is expected for a future time.

In one embodiment, the method includes reducing the electrical energy output of the radiofrequency generator such that heating factor at the future time is less than the predetermined threshold value.

In one embodiment, the method includes, in response to determining that the expected heating factor at the future time point is greater than the predetermined threshold value, outputting an alert.

In one embodiment, the expected heating factor for the future time point is determined based at least in part on a treatment plan, where the treatment plan includes discontinuing electrical energy flow from the radiofrequency generator for at least one period of time before restarting electrical energy flow.

In one embodiment, the electrical energy flow corresponds to current flow.

In one embodiment, the treatment plan includes discontinuing electrical energy flow from the radiofrequency generator for at least one period of time before restarting electrical energy flow.

In one embodiment, the treatment plan includes shutting down a particular radiofrequency probe of the at least one radiofrequency probe in response to determining that the particular radiofrequency probe has the highest electrical energy output.

In one embodiment, the method includes measuring, by an impedance sensor, a first impedance at the grounding pad for a first time and measuring, by the impedance sensor, a second impedance at the grounding pad for a second, later time.

In one embodiment, the method includes measuring disabling the at least one probe in response to determining that the second impedance differs from the first impedance by a value that exceeds a predetermined threshold value.

In one aspect, the present disclosure relates to a radiofrequency (RF) treatment system, which, in one embodiment, includes: a programmable controller configured to perform at least the following functions: measuring an electrical energy flow through a grounding pad, where the grounding pad is in contact with at least a portion of skin of a subject's skin, and where the electrical energy is delivered to a treatment site of the subject, from a radiofrequency (RF) generator by at least one radiofrequency probe; determining, based on the measured electrical energy through the grounding pad, a heating factor, where the heating factor represents a measure of heat absorbed by the subject over a predetermined period of time in response to the delivery of the electrical energy to the subject by the at least one radiofrequency probe; comparing the heating factor to a predetermined threshold value associated with damage to the subject outside of the treatment site, and in response to determining, from the comparison, that the heating factor is approaching or greater than the predetermined threshold value, performing at least one of: disabling the radiofrequency generator for at least a predetermined period of time; or increasing or decreasing the electrical energy flow, such that the controller regulates the electrical energy flow in accordance with a closed loop feedback system.

In one embodiment, the heating factor is a total heating factor that represents a running average of a plurality of incremental heating factors.

In one embodiment, the plurality of incremental heating factors are recorded at regular intervals.

In one embodiment, the system includes: measuring the electrical energy flow through the radiofrequency probe, where the at least one radiofrequency probe is in contact with at least a first portion of the skin of the subject at the treatment site; and disabling the at least one radiofrequency ablation probe when the measured electrical energy flow from the at least one radiofrequency ablation probe is different from the electrical energy flow through the grounding pad.

In one embodiment, the at least one radiofrequency probe is a plurality of radiofrequency probes that are in contact with a plurality of locations at the treatment site, and where a sum of the electrical energy flow through each of the radiofrequency probes is compared to the electrical energy flow through the grounding pad

In one embodiment, the controller is configured to: disable all of the radiofrequency probes in response to determining that the sum of the electrical energy flowing through the radiofrequency probes is different from the electrical energy flow through the grounding pad.

In one embodiment, the controller is configured to calculate an expected heating factor that is expected for a future time.

In one embodiment, the controller is configured to reduce the electrical energy output of the radiofrequency generator such that heating factor at the future time point is less than the predetermined threshold value.

In one embodiment, the controller is configured to, in response to determining that the expected heating factor for the future time is greater than the predetermined threshold value, output an alert.

In one embodiment, the expected heating factor at the future time is determined based at least in part on a treatment plan, where the treatment plan includes discontinuing electrical energy flow from the radiofrequency generator for at least one period of time before restarting electrical energy flow.

In one embodiment, the electrical energy flow corresponds to current flow.

In one embodiment, the treatment plan includes discontinuing electrical energy flow from the radiofrequency generator for at least one period of time before restarting electrical energy flow from the radiofrequency generator.

In one embodiment, the treatment plan includes shutting down a particular radiofrequency probe of the plurality of radiofrequency probes in response to determining that the particular radiofrequency probe has the highest electrical energy output.

In one embodiment the system includes an impedance sensor configured to measure a first impedance value at the grounding pad at a first time and measure a second impedance value at the grounding pad at a second, later time.

In one embodiment, the controller is configured to disable one or more of the at least one radiofrequency probe in response to determining that the second impedance value differs from the first impedance by at least a predetermined threshold difference.

Other aspects and features according to the example embodiments of the present disclosure will become apparent to those of ordinary skill in the art, upon reviewing the following detailed description in conjunction with the accompanying figures.

### BRIEF DESCRIPTION OF THE DRAWINGS

Reference will now be made to the accompanying drawings, which are not necessarily drawn to scale.

FIG. 1 is a diagram of a system for applying cooled radiofrequency (RF) electrical energy to target tissue in a subject's body, including a probe assembly and one or more cooling devices, and a grounding pad, according to some embodiments.

FIG. 2 is a diagram of a system for applying cooled RF electrical energy to a subject that includes a quad pump unit and is configured for a two-probe connection, according to some embodiments.

FIG. 3 is a diagram of a quad pump unit, according to some embodiments.

FIG. 4 is a diagram of a dual pump unit, according to some embodiments.

FIG. 5 is a flow chart of a method of determining a heating factor and controlling an RF electrical energy flow based on the heating factor, according to some embodiments.

FIG. 6 is a block diagram of a computing device for implementing the various methods described herein, according to some embodiments.

### DETAILED DESCRIPTION

In some aspects, the disclosed technology relates to systems, systems, devices and methods relate to radiofrequency treatment procedures. Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, which may be referred to herein as an “area of interest”, “region of interest”, or “target location”. For the purposes of the present disclosure, a lesion refers to any effect achieved through the application of energy to a tissue in a patient's body, and the disclosure is not intended to be limited in this regard. Furthermore, for the purposes of this description, proximal generally indicates that portion of a device or system next to or nearer to a user (when the device is in use), while the term distal generally indicates a portion further away from the user (when the device is in use).

Reference will now be made in detail to one or more embodiments of the present disclosure, examples of which are illustrated in the drawings. Each example and embodiment is provided by way of explanation of the disclosure, and is not meant as a limitation of the disclosure. For example, features illustrated or described as part of one embodiment may be used with another embodiment to yield still a further embodiment. It is intended that the disclosure include these and other modifications and variations as coming within the scope and spirit of the disclosure.

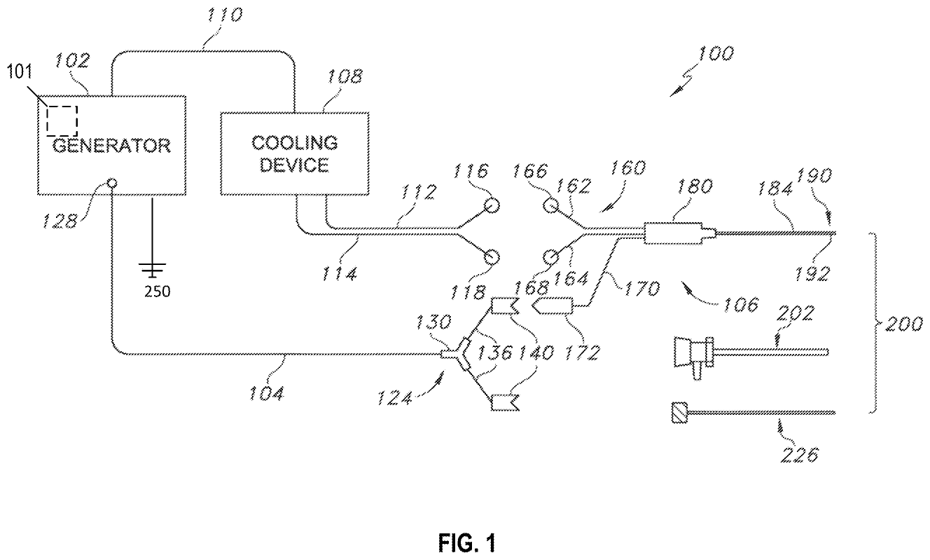

Generally speaking, the present disclosure in some embodiments is directed to a cooled radiofrequency ablation system. The system includes a probe assembly having a proximal region, a distal tip region, and a hollow elongated shaft. The hollow elongated shaft defines an internal cavity, and a first internal cooling fluid tube and a second internal cooling fluid tube are positioned inside the internal cavity and extend from the proximal region. Further, the distal tip region includes a conductive portion for delivering energy to a target location within tissue. The system also includes a radiofrequency generator for delivering energy to the target location within tissue via the conductive portion of the distal tip region of the probe assembly, as well as a cooling device including a cooling fluid reservoir and a bidirectional pump assembly operable to circulate a cooling fluid from the cooling fluid reservoir through the first internal cooling fluid tube, the internal cavity, the second internal cooling fluid tube, and back to the cooling fluid reservoir when the bidirectional pump is operating in a first direction; or from the cooling fluid reservoir through the second internal cooling fluid tube, the internal cavity, the first internal cooling fluid tube, and back to the cooling fluid reservoir when the bidirectional pump is operating in a second direction. The various features of the cooled radiofrequency ablation system will now be discussed in more detail in reference to FIGS. 1-8.

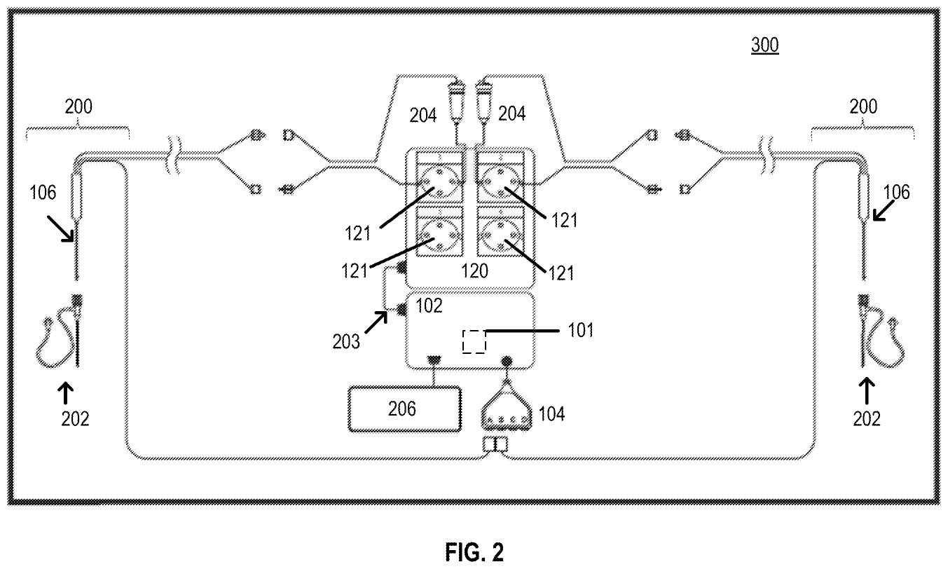

Turning first to FIG. 1, a schematic diagram of an energy delivery system 100 for the delivery of energy, such as RF energy, to a target location of tissue of a patient is provided and is presented herein for purposes of describing an exemplary operating environment in which the present introducer and assembly may be used. The system 100 includes a generator 102, a cable 104, one or more probe assemblies 106 (only one probe assembly is shown), one or more cooling devices 108 that include a one or more cooling fluid reservoirs (not shown) and a bidirectional pump assembly 120 (see FIG. 2), a pump cable 110, one or more proximal cooling fluid supply tubes 112, and one or more proximal cooling fluid return tubes 114. Throughout the present disclosure, the term “pump assembly” may be used interchangeably with “pump unit.” The generator 102 may be a radiofrequency (RF) generator, or any other energy source, such as microwave energy, thermal energy, ultrasound, or optical energy. The generator 102 may be connected to a display that displays various visual representations related to a treatment procedure, such as any parameters that are relevant to a treatment procedure, for example temperature, impedance, etc. and errors or warnings related to a treatment procedure. Alternatively, the generator 102 may include means of transmitting a signal to an external display. The generator 102 is operable to communicate with the first and second probe assemblies 106 and the one or more cooling devices 108. Such communication may be unidirectional or bidirectional depending on the devices used and the procedure performed.

In addition, as shown, a distal region 124 of the cable 104 may include a splitter 130 that divides the cable 104 into two distal ends 136 such that the probe assemblies 106 can be connected thereto. A proximal end 128 of the cable 104 is connected to the generator 102. This connection can be permanent, whereby, for example, the proximal end 128 of the cable 104 is embedded within the generator 102, or temporary, whereby, for example, the proximal end 128 of cable 104 is connected to generator 102 via an electrical connector. The two distal ends 136 of the cable 104 terminate in connectors 140 operable to couple to the probe assemblies 106 and establish an electrical connection between the probe assemblies 106 and the generator 102. In alternate embodiments, the system 100 may include a separate cable for each probe assembly 106 being used to couple the probe assemblies 106 to the generator 102.

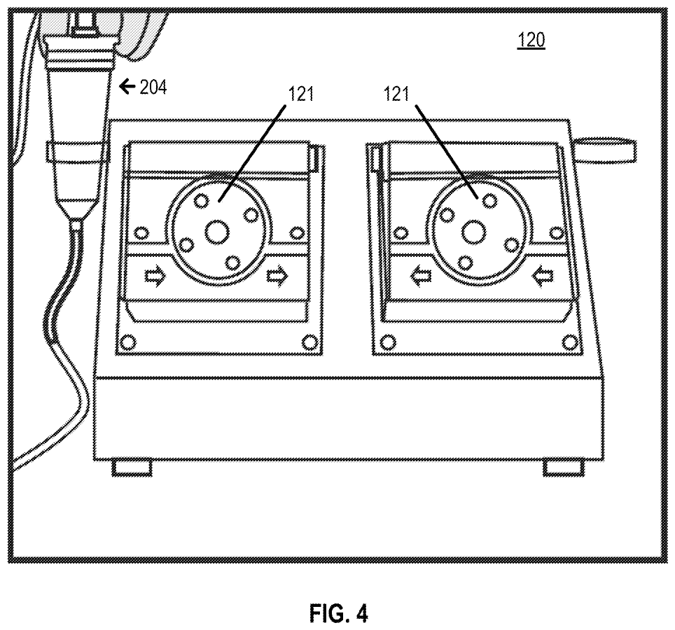

The cooling device(s) 108 may include any means of reducing a temperature of material located at and proximate to one or more of the probe assemblies 106. For example, the cooling devices 108 may include a pump assembly 120 (see FIG. 2), such as a bidirectional pump assembly, operable to circulate a fluid from the cooling devices 108 through one or more proximal cooling fluid supply tubes 112, the probe assemblies 106 (e.g., through an internal cavity 122 of the probe assemblies 106 (see FIGS. 4-7)), one or more proximal cooling fluid return tubes 114, and back to the one or more cooling devices 108.

The system 100 may include a programmable controller (which may also be referred to herein as simply a “controller”) for facilitating communication between the cooling devices 108 and the generator 102, via a feedback control loop. The feedback control may be implemented, for example, in a control module which may be a component of the generator 102. In such embodiments, the generator 102 is operable to communicate bidirectionally with the probe assemblies 106 as well as with the cooling devices 108, wherein bidirectional communication refers to the capability of a device to both receive a signal from and send a signal to another device.

As an example, the generator 102 may receive temperature measurements from one or both of the first and second probe assemblies 106. Based on the temperature measurements, the generator 102 may perform some action, such as modulating the power that is sent to the probe assemblies 106. Thus, both probe assemblies 106 may be individually controlled based on their respective temperature measurements.

The pumps associated with the cooling devices 108 may communicate a fluid flow rate to the generator 102 and may receive communications from the generator 102 instructing the pumps to modulate this flow rate. With the cooling devices 108 turned off, any temperature sensing elements associated with the probe assemblies 106 would not be affected by the cooling fluid allowing a more precise determination of the surrounding tissue temperature to be made. In addition, when using more than one probe assembly 106, the average temperature or a maximum temperature in the temperature sensing elements associated with probe assemblies 106 may be used to modulate cooling. The cooling devices 108 may reduce the rate of cooling or disengage depending on the distance between the probe assemblies 106. For example, when the distance is small enough such that a sufficient current density exists in the region to achieve a desired temperature, little or no cooling may be required. In such an embodiment, energy is preferentially concentrated between first and second energy delivery devices 192 through a region of tissue to be treated, thereby creating a strip lesion characterized by an oblong volume of heated tissue that is formed when an active electrode is in close proximity to a return electrode of similar dimensions.

The cooling devices 108 may also communicate with the generator 102 to alert the generator 102 to one or more possible errors and/or anomalies associated with the cooling devices 108. For example, if cooling flow is impeded or if a lid of one or more of the cooling devices 108 is opened. The generator 102 may then act on the error signal by at least one of alerting a user, aborting the procedure, and modifying an action. Still referring to FIG. 1, the proximal cooling fluid supply tubes 112 may include proximal supply tube connectors 116 at the distal ends of the one or more proximal cooling fluid supply tubes 112. Additionally, the proximal cooling fluid return tubes 114 may include proximal return tube connectors 118 at the distal ends of the one or more proximal cooling fluid return tubes 114. In one embodiment, the proximal supply tube connectors 116 are female Luer-lock type connectors and the proximal return tube connectors 118 are male Luer-lock type connectors although other connector types are intended to be within the scope of the present disclosure. In addition, as shown in FIG. 1, the probe assembly 106 may include a proximal region 160, a handle 180, a hollow elongate shaft 184, which can also be referred to as an electrocap, and a distal tip region 190 that includes the one or more energy delivery devices 192 and that can also be referred to as the active tip. The elongate shaft 184 may be manufactured out of polyimide, which provides exceptional electrical insulation while maintaining sufficient flexibility and compactness. In alternate embodiments, the elongate shaft 184 may be any other material imparting similar qualities. In still other embodiments, the elongate shaft 184 may be manufactured from an electrically conductive material and may be covered by an insulating material so that delivered energy remains concentrated at the energy delivery device 192 of the distal tip region 190. The proximal region 160 includes a distal cooling fluid supply tube 162, a distal supply tube connector 166, a distal cooling fluid return tube 164, a distal return tube connector 168, a probe assembly cable 170, and a probe cable connector 172. In such embodiments, the distal cooling fluid supply tube 162 and distal cooling fluid return tube 164 are flexible to allow for greater maneuverability of the probe assemblies 106, but alternate embodiments with rigid tubes are possible.

The distal supply tube connector 166 may be a male Luer-lock type connector and the distal return tube connector 168 may be a female Luer-lock type connector. Thus, the proximal supply tube connector 116 may be operable to interlock with the distal supply tube connector 166 and the proximal return tube connector 118 may be operable to interlock with the distal return tube connector 168.

The probe cable connector 172 may be located at a proximal end of the probe assembly cable 170 and may be operable to reversibly couple to one of the connectors 140, thus establishing an electrical connection between the generator 102 and the probe assembly 106. The probe assembly cable 170 includes one or more conductors to transmit RF current from the generator 102 to the one or more energy delivery devices 192, as well as to connect multiple temperature sensing devices to the generator 102 as discussed below.

The energy delivery devices 192 may include any means of delivering energy to a region of tissue adjacent to the distal tip region 190. For example, the energy delivery devices 192 may include an ultrasonic device, an electrode or any other energy delivery means and the invention is not limited in this regard. Similarly, energy delivered via the energy delivery devices 192 may take several forms including but not limited to thermal energy, ultrasonic energy, radiofrequency energy, microwave energy or any other form of energy. For example, in one embodiment, the energy delivery devices 192 may include an electrode. The active region of the electrode may be 2 to 20 millimeters (mm) in length and energy delivered by the electrode is electrical energy in the form of current in the RF range. The size of the active region of the electrode can be optimized for placement within an intervertebral disc, however, different sizes of active regions, all of which are within the scope of the present invention, may be used depending on the specific procedure being performed. In some embodiments, feedback from the generator 102 may automatically adjust the exposed area of the energy delivery device 192 in response to a given measurement such as impedance or temperature. For example, in one embodiment, the energy delivery devices 192 may maximize energy delivered to the tissue by implementing at least one additional feedback control, such as a rising impedance value.

FIG. 1 also depicts an introducer 202 and a stylet 226, wherein the combination of the RF probe assembly 106, the introducer 202, and the stylet 226 define an RF ablation probe system 200 in accordance with aspects of the present invention.

FIG. 2 illustrates a system capable of performing a cooled radiofrequency (RF) treatment procedure. According to some embodiments of the present disclosure, the system includes a pump assembly 120 and a generator 102. The pump 120 assembly can include different numbers of pump heads 121. Different numbers of pump heads 121 can be used in different embodiments of the present disclosure, in combinations with different numbers of probe systems 200. The pump heads 121 can be configured to move a cooling fluid through one or more tubes connected to a medical probe system 200.

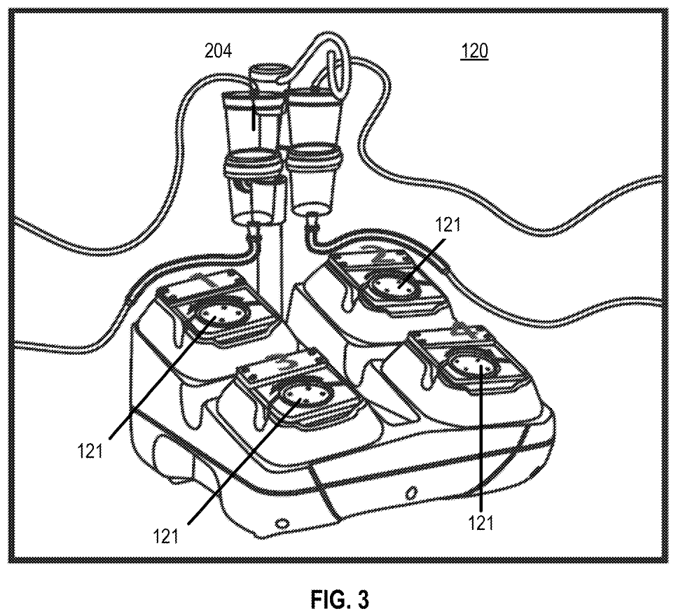

The present disclosure contemplates that any number of pump heads 121 can be used in the pump assembly 120. In some embodiments of the present disclosure, the pump assembly 120 includes two pump heads 121 (a “dual pump unit”). In other embodiments of the present disclosure, the pump assembly 120 includes four pump heads 121 (a “quad pump unit”). A perspective view of a quad pump unit is shown in FIG. 3, and a front-view of a dual-pump unit is shown in FIG. 4. In some embodiments of the present disclosure, the pump heads 121 can be peristaltic pump heads 121, as shown in FIGS. 3 and 4. Tubing can be connected to the peristaltic pump heads 121 by inserting the tubing into the peristaltic pump heads 121. In some embodiments of the present disclosure, each pump head 121 includes a motor, which drives the pump head 121, pumping the cooling fluid through the tubing for a cooled RF treatment procedure.

As shown in the embodiment of the present disclosure shown in FIG. 2, the system 300 can include two probe systems 200 and a grounding pad 206. The pump heads 121 of the pump assembly 120 move cooling fluid through tubing and to the probe assemblies 106. In the embodiment shown in FIG. 2, the pump assembly 120 is a four-pump pump unit (“quad pump unit” or “QPU”). The cooling fluid can exit through a second length of tubing and a burette 204 to return to one of the pump heads 121 of the pump assembly 120. In some embodiments of the present disclosure, each pump head 121 is configured to supply cooling fluid to one probe assembly 106 although it is contemplated that in other embodiments the numbers of probe assemblies 106 and pump heads 121 can be different. For example, in the embodiment of the present disclosure shown in FIG. 2, the pump assembly 120 includes four pump heads 121 but only two are active (shown as not connected to the probe assemblies 201 or burettes 204).

The grounding pad 206 can be placed in contact with a portion of the patient's skin (not shown). A current sensor (not shown) can be configured to measure the current flow through the grounding pad 206, and the current sensor can be operably connected to the generator 102 or the controller 101. The controller 101 can be configured to measure and record the current flow through the grounding pad 206, and to log the current flow through the grounding pad 206 over a period of time. Additionally, an impedance sensor (not shown) can be configured to measure the impedance of the subject's skin, the grounding pad 206, or a combination of the subject's skin and grounding pad 206.

It is also contemplated by the present disclosure that the impedance, current, and/or voltage can be calculated based on other values measured. As a non-limiting example, the impedance can be determined by measuring the voltage across the grounding pad 206 and current through the grounding pad 206 and thereby calculating the impedance. Other techniques for determining voltage, current, and impedance will are contemplated by the present disclosure.

Additional configurations of probe assemblies 106 are contemplated by the present disclosure, and the two-probe configuration shown in FIG. 2 is intended only as a non-limiting example. Non-limiting examples of other probe configurations include one-probe, three probe, and four probe configurations.

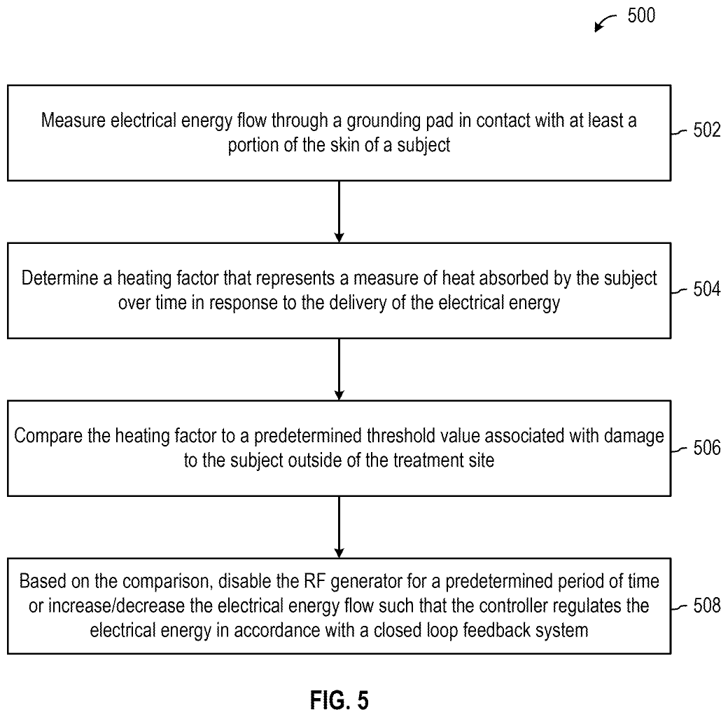

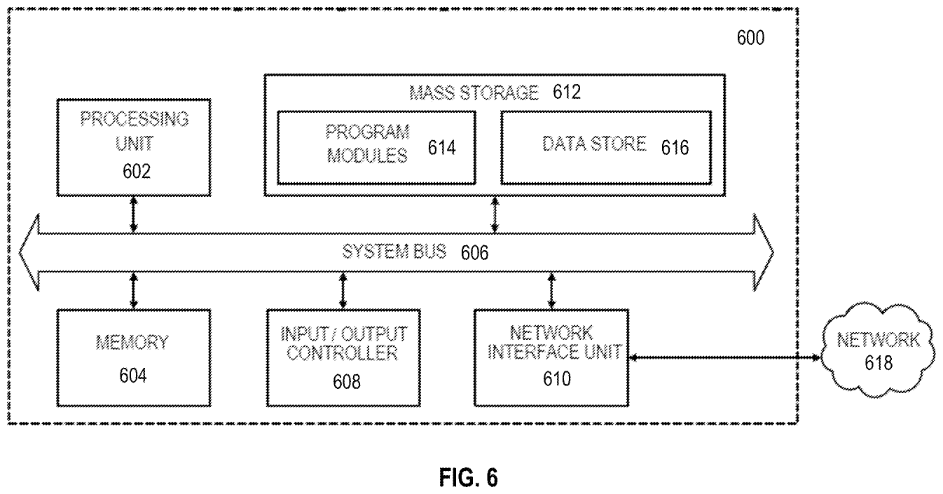

The generator 102, controller 101, and/or pump assembly 120 can be configured to implement the method disclosed by FIG. 5, described in further detail below. In some embodiments of the present disclosure, the generator 102 and/or pump assembly 120 can include one or more components of the computer 600 shown in FIG. 6. An electrical connection 203 between the generator 102 and pump unit 120 can be used to transmit sensor information between the generator 102 and pump unit 120. The electrical connection 203 can also be used by the generator 102 and/or pump assembly 120 to send control signals to each other. Additionally, instructions and user inputs can pass from the generator 102 to the pump unit 120 or vice versa. In the embodiment depicted in FIG. 2, the controller 101 is part of the generator 102. The generator 102 and/or controller 101 can determine information identifying the pump assembly 120 using resistor ID, device ID stored in a memory unit on a computing device within the pump (e.g., a computing device 600) by a network connection (e.g., SSL, Bluetooth, Wi-Fi) or by a serial communication signal (e.g., I2C, Serial/RS232). Alternatively, or additionally, the generator 102 and/or controller 101 can determine identifying information related to for some or all of the motors and/or motor controllers included in the pump assembly 120. In some embodiments of the present disclosure, each motor that is part of the pump assembly 120 includes a separate motor controller corresponding to that motor, and the motor controllers are separate controllers from the controller 101. However, it should be understood that the present disclosure contemplates that the motor controllers can be integrated into the generator 102, controller 101, or that one motor controller can be used for multiple or all of the motors that are part of the pump assembly 120.

FIG. 5 illustrates a method for radiofrequency treatment, according to one embodiment of the present disclosure. The embodiment of the present disclosure illustrated in FIG. 5 can be implemented using the systems illustrated in FIGS. 1-4 of the present disclosure, although it should be understood that the method illustrated in FIG. 5 could be used with embodiments of the present disclosure other than those shown in FIGS. 1-4. The steps illustrated in FIG. 5 can be performed using a programmable controller 101, for example the computing device 600 illustrated in FIG. 6. Similarly, the grounding pad 206 can be the grounding pad 206 illustrated in FIGS. 1 and 2.

As shown in FIG. 5, in one embodiment of the present disclosure, the controller 101 can be configured to measure 502 an electrical energy flow (e.g., an electrical flow) through a grounding pad 206. In some embodiments of the present disclosure, the grounding pad 206 is a conductive pad that is configured to adhere to the subject's skin. The grounding pad 206 can be connected to one or more wires are connected to an electrical ground. The grounding pad completes the electrical circuit for the cooled RF procedure where the probes 106 deliver the electrical current, and the return path for the electrical current is provided by the grounding pad. The probes 106, in turn, receive electrical energy from the generator 102. It should be understood that, in the present disclosure, the “grounding pad” 206 can refer to any structure that serves to electrically ground the subject, and other terms in the art for grounding pads include “dispersive electrode” and “neutral electrode.”

Measuring 502 the electrical energy flow through the grounding pad can include sensing the current flow through the grounding pad 206, for example using a current sensor.

Based on the measured electrical energy flow through the grounding pad 206 a heating factor can be determined 504. The heating factor can represent the heat absorbed by the subject and can be proportional to the total electrical energy absorbed by the subject over a period of time. The heating factor can be based on, for example, the current delivered and the time period in which that current was delivered. Additionally, the heating factor can be proportional to an actual temperature rise in the subject.

A non-limiting example of a heating factor that can be calculated in embodiments of the present disclosure is a heating actor based on the electrical current squared multiplied by the activation time:

$$Heating Factor = ( {I}^{⋀} 2 ) * ( activation time )$$

Another non-limiting example of a method to calculate heating factor that can be used in some embodiments of the present disclosure is a method that calculates an incremental heating factor. This method of determining a heating factor can be used in situations where the current (e.g., the electrical energy flow from a generator 102 or probes 106) is varied in a time period, and it is desired to determine the heating factor over that time period. A number of incremental heating factors can be calculated for predetermined time periods, and those incremental heating factors can be summed to obtain a “running total heating factor.” This running total heating factor can be based on the sum of the incremental heating factors a specified time period. Values outside the specified running total time period (i.e. values that are too old) can be excluded from the calculation of the running total time period.

As a non-limiting example, the predetermined time period for the incremental heating factors can be 20 milliseconds (ms), and the running total time period can be 60 seconds. So at the end of the 20 ms time periods, the oldest measured heating factor is removed from the total, and a new calculation the heating factor is calculated for the 20 ms time period is added to the total. This can create a running total heating factor including the individual heating factors calculated for each of the 20 ms incremental heating factors. According to one embodiment, the heating factor of each individual incremental heating factor is:

$$Incremental Heating Factor = ( {I}^{⋀} 2 ) * ( 20 milliseconds )$$

According to this embodiment, the running total heating factor is the sum of all the incremental heating factors recorded over the previous 60 seconds, and the current (I) is the current during the 20 millisecond incremental time periods.

Again, it should be understood that the above formulas and time periods are intended only as non-limiting examples, and that other formulas and time periods are contemplated by the present disclosure. The present disclosure contemplates that other measurements and definitions of “heating factor” can be used, and the heating factor calculations provided herein are intended only as nonlimiting examples. For example, the present disclosure contemplates that the heating factor can be calculated using smaller incremental time periods, or that the running total heating factor can be calculated continuously.

The heating factor can be compared 506 to a predetermined threshold value. In some embodiments of the present disclosure, the predetermined threshold value can be determined based on experimental data. For example, the predetermined threshold value can be a heating factor value that is associated with damage to the subject outside the treatment site. Additionally, the predetermined threshold value can be determined according to a user input, or according to the procedure that the generator 102 and/or controller 101 are configured to perform.

If the heating factor is approaching the predetermined threshold value or greater than the predetermined threshold value, the controller 101 can disable the radiofrequency generator 102, and/or increase or decrease the electrical energy flow. In some embodiments of the present disclosure, the radiofrequency generator 102 is disabled until the heating factor (for example a running total heating factor) is below a predetermined threshold. According to some embodiments of the present disclosure, the radiofrequency generator 102 can be disabled for a predetermined period of time. The predetermined period of time can be a period of time based on the heating factor, the treatment type, or other characteristics of the system 100 300 used in the procedure.

According to some embodiments of the present disclosure, the electrical energy flow is increased or decreased based on the heating factor. If the heating factor is below the predetermined threshold, the electrical energy flow can be increased to raise the heating factor to the predetermined threshold, or another target amount. Conversely, if the heating factor is above the predetermined threshold, the electrical energy flow can be decreased so that the heating factor decreases to the predetermined threshold, or a point below the predetermined threshold.

It is also contemplated by the present disclosure that the controller 101 can calculate a rate of change of the heating factor and perform a control loop based on that rate of change. The control loop can be a closed loop feedback system. As a non-limiting example, the controller 101 can detect that the heating factor is increasing toward the predetermined threshold, and reduce the electrical energy flow from the generator 102 so that the heating factor reaches the predetermined threshold without exceeding it.

Some embodiments of the present disclosure can predict an expected heating factor at a future time point. For example, the controller 101 can estimate the heating factor at a future point in time based on the electrical energy flow through the probes. This information can be displayed to a user using a graphical user interface or other suitable display. If the estimated heating factor at a future point in time is greater than the predetermined threshold value, an alert can be displayed to the user. In some embodiments of the present disclosure, the predicted heating factor at a future time point can be the predicted running total heating factor. In some embodiments of the present disclosure the controller 101 can use the predicted heating factor as part of the closed loop feedback system or other control loop.

Similarly, in some embodiments of the present disclosure, the controller 101 can reduce the electrical energy output of the radiofrequency generator 102 such that the heating factor at the future time point is less than a predetermined threshold value.

In some embodiments of the present disclosure, an estimated heating factor at a future point in time can be based on a treatment plan. The treatment plan can include information about the treatment being performed. Non-limiting examples of the kinds of information that can be included in the treatment plan include information about the number of probes used and the characteristics of the probes used, the size and characteristics of the grounding pad, and the amount of electrical energy that will be delivered through the probes at different times in the treatment process. The controller 101 can estimate the expected heating factor at different points in time during the treatment plan and display that information to a user. Similarly, in some embodiments of the present disclosure, an alert can be displayed when the expected heating factor during the treatment plan exceeds a predetermined threshold.

In some embodiments of the present disclosure, the controller 101 can disable the generator 102 for a period of time during the treatment plan. As a non-limiting example, the generator 102 can be disabled until the heating factor drops to predetermined threshold, or the generator 102 can be disabled until the heating factor drops to a point below the predetermined threshold.

Some embodiments of the present disclosure can include impedance sensors configured to calculate the impedance of the grounding pad. Similarly, some embodiments of the present disclosure can be configured to measure an impedance or a change in impedance based on other measured values (e.g., voltage and current). The impedance can be measured at one or more time intervals.

In some embodiments of the present disclosure, the controller 101 can disable the generator 102 when a change in impedance between two time intervals exceeds a predetermined amount. In some embodiments of the present disclosure, one or more of the probes is disabled in addition to or instead of disabling the generator 102. In some embodiments of the present disclosure, the controller 101 can reduce the electrical energy output of the generator 102 based on the impedance. According to some embodiments of the present disclosure, an alert can be displayed to the user indicating that the impedance has changed by more than the predetermined amount. As a non-limiting example, a change in impedance can indicate that the grounding pad 206 is not fully contacting the subject. When the area of the grounding pad contacting the subject decreases, the same current flows through a smaller area, increasing the impedance between the grounding pad and the skin, and increasing the amount of heat that is produced in that location.

In some embodiments of the present disclosure, multiple systems 100 300 can be used, including, for example, multiple generators 102 and controllers 101. The heating factor for the combined systems 100 300 can be measured and one or more of the systems can be disabled when the heating factor exceeds a predetermined threshold. As a non-limiting example, the radiofrequency generator 102 with the highest electrical energy output can be disabled while the heating factor is higher than a predetermined threshold.

In some embodiments of the present disclosure, the controller 101 can change the duty cycle of the probes based on the heating factor. The duty cycle can represent the percentage of the time that the probes are enabled or disable. It should be understood that the control loop and closed loop control methods described above can be applied to controlling the duty cycle of the RF probes 106 based on the heating factor. As a non-limiting example, the duty cycle can be controlled so that the heating factor remains at or below a predetermined threshold.

According to some embodiments of the present disclosure, the controller 101 can implement a PID (proportional-integral-derivative) control loop. The PID control loop can control the power output of one or more probes 106 based on the heating factor or running total heating factor. Similarly, the controller 101 can calculate a total heating factor using an integral to represent or calculate the heating factor for a time period.

Some embodiments of the present disclosure can be configured to disable only one probe or disable the probes in sequence. For example, in a procedure where two probes are used, one probe 106 can be disabled to reduce the heating factor over time, while another probe 106 can remain enabled until a treatment process is complete. When the enabled probe 106 completes the treatment process, the disabled probe can be re-enabled.

It should also be understood that the controller 101 can control other parts of the system 100, 300 based on the heating factor. For example, the speed of the pump heads can be controlled. In some embodiments of the present disclosure, reducing the speed of the pump heads can reduce the current/power delivered without reducing the temperature of the probe.

It should also be understood that the techniques described herein can be used in combination in some embodiments of the present disclosure. As a non-limiting example, in a four-probe procedure where the heating factor exceeds the predetermined threshold, the controller 101 can disable one probe, change the duty cycle of another probe, and reduce the current output by another probe, while the remaining probe is unaffected. Similarly, the alerts described above can be combined, for example, the system can provide alerts that the heating factor is approaching the predetermined threshold at the same time as providing an alert that the treatment plan will cause the heating factor to exceed that predetermined threshold at another point in time.

Finally, it should be understood that the above methods can be used in combination with temperature sensors located on the grounding pad 206. For example, a heat sensor can be positioned on the grounding pad, and the system can use the temperature data from the heat sensor as part of the control loop to determine when to disable the probes. As a non-limiting example, the generator 102 and/or probes can be disabled when the heat of the pad exceeds a certain temperature, regardless of the heating factor.

In some embodiments of the present disclosure, the electrical energy flow through the radiofrequency probes can be measured. The sum of the electrical energy flows through the radiofrequency probes can be compared to the electrical energy flow through the grounding pad. Because electrical energy flows from the radiofrequency probes to the grounding pad through the subject, it is expected that the current flow from the radiofrequency probes should match the current flow through the grounding pad. In some embodiments of the present disclosure, the controller 101 can display a warning or disable one or more of the radiofrequency probes when the electrical energy flow through the radiofrequency probes is not equal to the electrical energy flow through the grounding pad.

FIG. 6 is a computer architecture diagram showing a general computing system capable of implementing one or more embodiments of the present disclosure described herein. A computer 600 may be configured to perform one or more functions associated with embodiments illustrated in one or more of FIGS. 1-5. The controller 101 and generator 102 can include one or more components of the computer 600. It should be appreciated that the computer 600 may be implemented within a single computing device or a computing system formed with multiple connected computing devices. For example, the computer 600 may be configured for a server computer, desktop computer, laptop computer, or mobile computing device such as a smartphone or tablet computer, or the computer 600 may be configured to perform various distributed computing tasks, which may distribute processing and/or storage resources among the multiple devices.

As shown, the computer 600 includes a processing unit 602, a system memory 604, and a system bus 606 that couples the memory 604 to the processing unit 602. The computer 600 further includes a mass storage device 612 for storing program modules. The program modules 614 may include modules executable to perform one or more functions associated with embodiments illustrated in one or more of FIGS. 1-5. The mass storage device 612 further includes a data store 616.

The mass storage device 612 is connected to the processing unit 602 through a mass storage controller (not shown) connected to the bus 606. The mass storage device 612 and its associated computer storage media provide non-volatile storage for the computer 600. By way of example, and not limitation, computer-readable storage media (also referred to herein as “computer-readable storage medium” or “computer-storage media” or “computer-storage medium”) may include volatile and non-volatile, removable and non-removable media implemented in any method or technology for storage of information such as computer-storage instructions, data structures, program modules, or other data. For example, computer-readable storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer 600. Computer-readable storage media as described herein does not include transitory signals.

According to various embodiments, the computer 600 may operate in a networked environment using connections to other local or remote computers through a network 618 via a network interface unit 610 connected to the bus 606. The network interface unit 610 may facilitate connection of the computing device inputs and outputs to one or more suitable networks and/or connections such as a local area network (LAN), a wide area network (WAN), the Internet, a cellular network, a radio frequency network, a Bluetooth-enabled network, a Wi-Fi enabled network, a satellite-based network, or other wired and/or wireless networks for communication with external devices and/or systems.

The computer 600 may also include an input/output controller 608 for receiving and processing input from a number of input devices. Input devices may include, but are not limited to, keyboards, mice, stylus, touchscreens, microphones, audio capturing devices, or image/video capturing devices. An end user may utilize such input devices to interact with a user interface, for example a graphical user interface on one or more display devices (e.g., computer screens), for managing various functions performed by the computer 600, and the input/output controller 608 may be configured to manage output to one or more display devices for visually representing data.

The bus 606 may enable the processing unit 602 to read code and/or data to/from the mass storage device 612 or other computer-storage media. The computer-storage media may represent apparatus in the form of storage elements that are implemented using any suitable technology, including but not limited to semiconductors, magnetic materials, optics, or the like. The program modules 614 may include software instructions that, when loaded into the processing unit 602 and executed, cause the computer 600 to provide functions associated with embodiments illustrated in FIGS. 1-5. The program modules 614 may also provide various tools or techniques by which the computer 600 may participate within the overall systems or operating environments using the components, flows, and data structures discussed throughout this description. In general, the program module 614 may, when loaded into the processing unit 602 and executed, transform the processing unit 602 and the overall computer 600 from a general-purpose computing system into a special-purpose computing system.

The processing unit 602 may be constructed from any number of transistors or other discrete circuit elements, which may individually or collectively assume any number of states. More specifically, the processing unit 602 may operate as a finite-state machine, in response to executable instructions contained within the program modules 614. These computer-executable instructions may transform the processing unit 602 by specifying how the processing unit 602 transitions between states, thereby transforming the transistors or other discrete hardware elements constituting the processing unit 602. Encoding the program modules 614 may also transform the physical structure of the computer-readable storage media. The specific transformation of physical structure may depend on various factors, in different implementations of this description. Examples of such factors may include, but are not limited to, the technology used to implement the computer-readable storage media, whether the computer-readable storage media are characterized as primary or secondary storage, and the like. For example, if the computer-readable storage media are implemented as semiconductor-based memory, the program modules 614 may transform the physical state of the semiconductor memory, when the software is encoded therein. For example, the program modules 614 may transform the state of transistors, capacitors, or other discrete circuit elements constituting the semiconductor memory.

As another example, the computer-storage media may be implemented using magnetic or optical technology. In such implementations, the program modules 614 may transform the physical state of magnetic or optical media, when the software is encoded therein. These transformations may include altering the magnetic characteristics of particular locations within given magnetic media. These transformations may also include altering the physical features or characteristics of particular locations within given optical media, to change the optical characteristics of those locations. Other transformations of physical media are possible without departing from the scope of the present disclosure.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. The patentable scope of certain embodiments of the disclosed technology is indicated by the appended claims and claims to be filed in non-provisional patent application(s) claiming priority to the present application, rather than the foregoing description.

1. A method for radiofrequency (RF) treatment, the method comprising:
measuring, by a controller, an electrical energy flow through a grounding pad, wherein the grounding pad is in contact with at least a portion of skin of a subject's skin, and wherein the electrical energy is delivered to a treatment site of the subject, from a radiofrequency (RF) generator by at least one radiofrequency probe;
determining, by the controller, based on the measured electrical energy through the grounding pad, a heating factor, wherein the heating factor represents a measure of heat absorbed by the subject over a predetermined period of time in response to the delivery of the electrical energy to the subject by the at least one radiofrequency probe;
comparing, by the controller, the heating factor to a predetermined threshold value associated with damage to the subject outside of the treatment site; and
in response to determining that the heating factor is approaching or greater than the predetermined threshold value, at least one of:
disabling, by the controller, the radiofrequency generator for at least a predetermined period of time; or
increasing or decreasing, by the controller, the electrical energy flow, such that the controller regulates the electrical energy flow in accordance with a closed loop feedback system.

2. The method of claim 1, wherein the heating factor is a total heating factor that represents a running average of a plurality of incremental heating factors.

3. The method of claim 2, wherein the plurality of incremental heating factors are recorded at regular intervals.

4. The method of claim 1, further comprising:
measuring, by the controller, the electrical energy flow through the at least one radiofrequency probe, wherein the at least one radiofrequency probe is in contact with at least a first portion of the skin of the subject at the treatment site; and
disabling, by the controller, the at least one radiofrequency ablation probe when the measured electrical energy flow from the at least one radiofrequency ablation probe is different from the electrical energy flow through the grounding pad.

5. The method of claim 2, wherein the at least one radiofrequency probe is a plurality of radiofrequency probes that are in contact with a plurality of locations at the treatment site, and wherein a sum of the electrical energy flow through each of the radiofrequency probes is compared to the electrical energy flow through the grounding pad, and the method further comprising:
disabling, by the controller, all of the radiofrequency probes in response to determining that the sum of the electrical energy flow through the radiofrequency probes is different from the electrical energy flow through the grounding pad.

6. The method of claim 1, further comprising calculating, by the controller, an expected heating factor that is expected for a future time.

7. The method of claim 6, further comprising reducing, by the controller, the electrical energy output of the radiofrequency generator such that the heating factor for the future time is less than the predetermined threshold value.

8. The method of claim 6, further comprising outputting, by the controller, an alert in response to determining that the expected heating factor for the future time is greater than the predetermined threshold value.

9. The method of claim 6, wherein the expected heating factor at the future time is determined based at least in part on a treatment plan, wherein the treatment plan includes discontinuing electrical energy flow from the radiofrequency generator for at least one period of time before restarting electrical energy flow.

10. The method of claim 9, wherein the electrical energy flow corresponds to current flow.

11. The method of claim 9, wherein the treatment plan includes discontinuing electrical energy flow from the radiofrequency generator for at least one period of time before restarting electrical energy flow.

12. The method of claim 11, wherein the treatment plan includes shutting down a particular radiofrequency probe of the at least one radiofrequency probe in response to determining that the particular radiofrequency probe has the highest electrical energy output.

13. The method of claim 4, further comprising:
measuring, by an impedance sensor coupled to the controller, a first impedance at the grounding pad for a first time; and
measuring, by the impedance sensor, a second impedance at the grounding pad at a second time that is later than the first time.

14. The method of claim 13, further comprising disabling, by the controller, one or more of the at least one radiofrequency probe in response to determining that the second impedance differs from the first impedance by a value that exceeds a predetermined threshold value.

15. A radiofrequency (RF) treatment system, the system comprising:
one or more processors; and
memory having instructions stored thereon that, when executed by the one or more processors, cause the system to:
measure an electrical energy flow through a grounding pad, wherein the grounding pad is in contact with at least a portion of skin of a subject's skin, and wherein the electrical energy is delivered to a treatment site of the subject, from a radiofrequency (RF) generator by at least one radiofrequency probe;
determine, based on the measured electrical energy through the grounding pad, a heating factor, wherein the heating factor represents a measure of heat absorbed by the subject over a predetermined period of time in response to the delivery of the electrical energy to the subject by the at least one radiofrequency probe;
compare the heating factor to a predetermined threshold value associated with damage to the subject outside of the treatment site; and
in response to determining, from the comparison, that the heating factor is approaching or greater than the predetermined threshold value, perform at least one of:
disabling the radiofrequency generator for at least a predetermined period of time; or
increasing or decreasing the electrical energy flow, such that the controller regulates the electrical energy flow in accordance with a closed loop feedback system.

16. The system of claim 15, wherein the heating factor is a total heating factor that represents a running average of a plurality of incremental heating factors.

17. The system of claim 16, wherein the plurality of incremental heating factors are recorded at regular intervals.

18. The system of claim 15, wherein the instructions further cause the system to:
measure the electrical energy flow through the at least one radiofrequency probe, wherein the at least one radiofrequency probe is in contact with at least a first portion of the skin of the subject at the treatment site; and
disable the at least one radiofrequency probe when the measured electrical energy flow from the at least one radiofrequency probe is different from the electrical energy flow through the grounding pad.

19. The system of claim 16, wherein the at least one radiofrequency probe is a plurality of radiofrequency probes that are in contact with a plurality of locations at the treatment site, and wherein a sum of the electrical energy flow through each of the radiofrequency probes is compared to the electrical energy flow through the grounding pad.

20. The system of any one of claim 19, wherein the instructions further cause the system to disable all of the radiofrequency probes in response to determining that the sum of the electrical energy flowing through the radiofrequency probes is different from the electrical energy flow through the grounding pad.

21. The system of claim 15, wherein the instructions further cause the system to calculate an expected heating factor that is expected for a future time.

22. The system of claim 21, wherein the instructions further cause the system to reduce the electrical energy output of the radiofrequency generator such that the heating factor for the future time is less than the predetermined threshold value.

23. The system of claim 21, wherein the instructions further cause the system to output an alert in response to determining that the expected heating factor for the future time is greater than the predetermined threshold value.

24. The system of claim 21, wherein the expected heating factor at the future time is determined based at least in part on a treatment plan, and wherein the treatment plan includes discontinuing electrical energy flow from the radiofrequency generator for at least one period of time before restarting electrical energy flow.

25. The system of claim 15, wherein the electrical energy flow corresponds to current flow.

26. The system of claim 24, wherein the treatment plan includes discontinuing electrical energy flow from the radiofrequency generator for at least one period of time before restarting electrical energy flow from the radiofrequency generator.

27. The system of claim 24, wherein the treatment plan includes shutting down a particular radiofrequency probe of the at least one radiofrequency probe in response to determining that the particular radiofrequency probe has the highest electrical energy output.

28. The system of claim 18, further comprising an impedance sensor configured to:
measure a first impedance value at the grounding pad at a first time; and
measure a second impedance value at the grounding pad at a second, later time.

29. The system of claim 28, wherein the instructions further cause the system to disable one or more of the at least one radiofrequency probe in response to determining that the second impedance value differs from the first impedance by at least a predetermined threshold amount.
